(19) United States
(12) Patent Application Publication
(10) Pub. No.: US 2024/0358493 A1
(43) Pub. Date: Oct. 31, 2024

(54) DEVICES, SYSTEMS AND METHODS FOR TREATING BENIGN PROSTATIC HYPERPLASIA AND OTHER CONDITIONS

(71) Applicant: Teleflex Life Sciences LLC, Wilmington, DE (US)

(72) Inventors: Theodore C. Lamson, Pleasanton, CA (US); Floria Cheng, San Francisco, CA (US); Joseph Catanese, III, Oakland, CA (US)

(73) Assignee: Teleflex Life Sciences LLC, Wilmington, DE (US)

(21) Appl. No.: 18/766,028

(22) Filed: Jul. 8, 2024

Related U.S. Application Data
(63) Continuation of application No. 18/181,914, filed on Mar. 10, 2023, now Pat. No. 12,042,372.
Application No. 18/181,914 is a continuation of application No. 17/224,996, filed on Apr. 7, 2021, now Pat. No. 11,850,140.
Application No. 17/224,996 is a continuation of application No. 16/234,282, filed on Dec. 27, 2018, now abandoned.
Application No. 16/234,282 is a continuation of application No. 13/830,811, filed on Mar. 14, 2013, now Pat. No. 10,195,014.

(51) Int. Cl. A61F 2/04; A61B 17/00; A61B 17/02; A61B 17/04; A61B 17/06; A61B 17/34; A61B 17/42; A61B 18/00; A61B 18/14; A61B 18/18; A61B 18/20; A61F 2/00

(52) U.S. Cl. CPC A61F 2/04; A61B 17/0401; A61B 17/0469; A61B 17/0482; A61B 17/0487; A61B 17/06109; A61B 17/42; A61F 2/0022; A61B 2017/00022; A61B 17/00234; A61B 2017/00274; A61B 2017/00792; A61B 2017/00796; A61B 2017/00805; A61B 17/0218; A61B 2017/0404; A61B 2017/0409; A61B 2017/0417; A61B 2017/0419; A61B 2017/0451; A61B 2017/0454; A61B 2017/0456; A61B 2017/0458; A61B 2017/0462; A61B 2017/0464; A61B 17/0467; A61B 2017/0488; A61B 2017/06052; A61B 2017/06176; A61B 17/3468; A61B 17/3478; A61B 2018/00547; A61B 18/14; A61B 18/1815; A61B 18/20; A61F 2002/047

(57) ABSTRACT

Extra-urethral implants and methods of use are disclosed. Implants can treat disorders or diseases of the prostate by, for example, enlarging the lumen of the prostatic urethra.

### CROSS-REFERENCE TO RELATED APPLICATIONS

This application is a continuation of U.S. application Ser. No. 18/181,914, filed Mar. 10, 2023, which is a continuation of U.S. application Ser. No. 17/224,996 filed Apr. 7, 2021, now U.S. Pat. No. 11,850,140, which is a continuation of U.S. application Ser. No. 16/234,282, filed Dec. 27, 2018, now abandoned, which is a continuation of U.S. application Ser. No. 13/830,811, filed Mar. 14, 2013, now U.S. Pat. No. 10,195,014, each of which is entitled “Devices, Systems and Methods for Treating Benign Prostatic Hyperplasia and Other Conditions,” and each of which is expressly incorporated herein by reference.

### BACKGROUND

The present invention relates generally to medical devices and methods and more particularly to devices, systems and methods for treating conditions wherein a tissue (e.g., the prostate gland) has a) become enlarged and/or b) undergone a change in form, position, structure, rigidity or force exertion with respect to another anatomical structure and/or c) has begun to impinge upon or compress an adjacent anatomical structure (e.g., the urethra).

Benign Prostatic Hyperplasia (BPH) is one of the most common medical conditions that affect men, especially elderly men. It has been reported that, in the United States more than half of all men have histopathologic evidence of BPH by age 60 and, by age 85, approximately 9 out of 10 men suffer from the condition. Moreover, the incidence and prevalence of BPH are expected to increase as the average age of the population in developed countries increases.

Despite extensive efforts in both the medical device and pharmaco-therapeutic fields, current treatments remain only partially effective and are burdened with significant side effects. Certain devices used to displace urethral tissue, such as urethral stents, can become encrusted due to exposure to urine. This encrustation is an undesirable and problematic side effect.

Thus, there remains a need for the development of new devices, systems and methods for treating BPH as well as other conditions in which one tissue or anatomical structure impinges upon or compresses another tissue or anatomical structure.

### SUMMARY

Certain embodiments related to a system for enlarging a lumen of a prostatic urethra. The system includes a delivery tool and an implant carried by the delivery tool. The implant is shaped to at least partially circumscribe the prostatic urethra of a patient. The system also includes a depth guide. The depth guide and delivery tool cooperate to deploy the implant within the peri-urethral space and thereby enlarge the lumen of the prostatic urethra.

In some embodiments, the delivery tool has a sharp surface configured to penetrate the urethral wall. In some embodiments, delivery tool delivers energy to prostatic tissue. In some embodiments, the implant has a sharp surface configured to penetrate the urethral wall. In some embodiments, the implant is carried externally to at least part of the delivery tool. In some embodiments, the implant is carried internally to at least part of the delivery tool. In some embodiments, the system includes a pusher coupled to the implant. In some embodiments, the system includes a locking mechanism coupled to at least one of the delivery tool, the implant, or the pusher. In some embodiments, the implant includes a first section and a second section, and the first section is comparatively more flexible than the second section. In some embodiments, the implant is self expanding. In some embodiments, the delivery tool and the implant each have a radius of curvature and the delivery tool radius of curvature is greater than the implant radius of curvature. In some embodiments, the implant includes a first section and a second section, and the first section is frictionally-engaged with the second section. In some embodiments, the implant is configured to be deployed by overcoming the frictional engagement.

### BRIEF DESCRIPTION OF THE DRAWINGS

FIGS. 1A and 1B illustrate views of an embodiment in which a depth guide facilitates the delivery of an implant.

FIG. 2 illustrates views of an embodiment in which transmitters and receivers help determine the position of an implant.

FIG. 3 illustrates the use of an embodiment in which the implant transmits light that helps determine its location within tissue.

FIGS. 4A through 4E illustrate views of the challenges of implanting a device that straightens a naturally bent urethra.

FIG. 5 illustrates a view of a delivery tool and implant in which the implant rides on the outside of the delivery tool according to an embodiment. The delivery tool is stiff and sharp as compared to the implant.

FIG. 6 illustrates a view of a delivery tool and implant in which the implant is contained within the delivery tool according to an embodiment. The delivery tool is stiff and sharp as compared to the implant.

FIGS. 7A through 7C illustrate views of a method for placing an implant that is contained within a delivery tool according to an embodiment. A pusher and locking mechanisms facilitate delivery of the implant.

FIGS. 8A through 8C illustrate views of an embodiment of an implant with longitudinal flexibility to accommodate urethral anatomy.

FIG. 9 depicts an embodiment in which the impingement of further hyperplasia on the urethral lumen is limited.

FIGS. 10A through 10C illustrate views of an embodiment in which an implant is placed that provides radial force without significant longitudinal displacement.

FIGS. 11A through 11C illustrate views of an embodiment in which the implant expands after being placed by a delivery tool. The implant is carried within the delivery tool.

FIGS. 12A through 12B illustrate views of an implant that is frictionally connected to itself and deployed using a dilating member according to an embodiment.

FIG. 13 illustrates views of an embodiment in which the delivery tool has a tighter radius than the implant.

FIGS. 14A through 14C illustrative views of an embodiment in which the urethral lumen is enlarged prior to delivery of the implant.

FIGS. 15A through 15C illustrate views of an embodiment in which the implant is placed using an expanding delivery member that is expanded within the bladder.

FIG. 16 illustrates a view of a supra pubic implant delivery method according to an embodiment.

FIGS. 17A through 17C illustrative views of an arcuate implant and its delivery method according to an embodiment.

FIG. 18 illustrates a view of a self-cutting ring-type implant according to an embodiment.

FIG. 19 illustrates a view of a triangular implant according to an embodiment.

FIG. 20 illustrates a view of an implant consisting of nested rings according to an embodiment.

FIG. 21 illustrates a view of a series of implants connected by a flexible spine according to an embodiment.

### DETAILED DESCRIPTION

The following description of the preferred embodiments of the invention is not intended to limit the invention to these preferred embodiments, but rather to enable any person skilled in the art to make and use this invention. Disclosed herein are systems and methods for treating conditions wherein a tissue (e.g., the prostate gland) has a) become enlarged and/or b) undergone a change in form, position, structure, rigidity or force exertion with respect to another anatomical structure and/or c) has begun to impinge upon or compress an adjacent anatomical structure (e.g., the urethra).

Mechanically displacing prostatic tissue so as to enlarge the lumen of the prostatic urethra is an attractive long-term solution to BPH. However, as described above, chronic exposure of an implant to urine is undesirable. According to embodiments described herein, implants placed near the urethral boundary of the prostate can provide the mechanical forces necessary to enlarge the lumen of the prostatic urethra while avoiding chronic exposure to urine.

For the purposes of this application, the area of the prostate gland near the urethral surface can be referred to as the extra-urethral portion of the prostate. The extra-urethral portion of the prostate is also that portion of the prostate near enough to the urethral boundary such that the prostatic tissue is comparatively less spongy than the central part of the gland. The extra-urethral portion has sufficient mechanical integrity to hold an implant substantially in place. Because the size, shape, and tissue properties of the prostate can vary significantly from one subject to another, this application defines the extra-urethral region in terms of an approximate position relative to the prostatic urethra and in terms of the mechanical properties of the region. The extra-urethral region may also include, or be referred to as, the urethral wall without being limited exclusively to the membrane layer of the prostate immediately adjacent the prostatic urethra. The extra-urethral region may also include, or be referred to as, the peri-urethral region or pen-urethral tissue. Of course, in this application peri-urethral tissue still refers to the region of the urethra within the prostate.

Certain embodiments described herein place implants in the prostate by puncturing, cutting, dissecting, or otherwise penetrating the extra-urethral region of the prostate. In doing so, it is important to avoid puncturing the anatomy in undesirable locations, such as the urethral sphincter, the bladder, and ejaculation ducts. In certain embodiments, more than one extra-urethral implant is desirable to avoid puncturing such locations in the anatomy. For example, multiple implants could be placed such that one implant is distal to the ejaculation ducts and another implant is proximal to the ejaculation ducts. In this way, multiple implants can be used to provide the necessary mechanical dilation of and long-term stability in the urethral lumen while not substantially damaging sensitive parts of the local anatomy.

In certain embodiments, extra-urethral implants include cutting surfaces to facilitate delivery of the implant into tissue. Other surfaces of any delivery device used may be comparatively blunt such that the cutting is focused at a certain surface of the implant.

In certain embodiments, a delivery tool, member, and/or surface is used to cut, penetrate, dissect, separate, or otherwise provide a point of entry and optionally a path through tissue for an implant. In such embodiments, the delivery tool, member, and/or surface can be sharp, pointed, serrated, or otherwise configured to cut tissue. Further, the delivery tool, member, and/or surface can be configured to instead, or in addition, delivery energy (e.g., radio frequency, ultrasound, and/or laser) to tissue to accomplish the penetration.

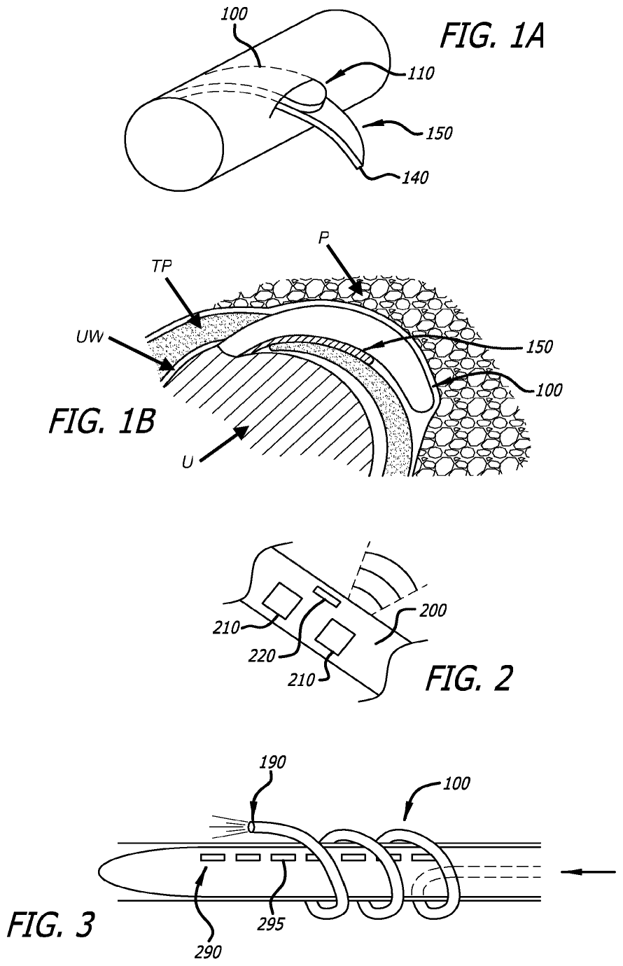

In certain embodiments, it is preferable to use selective blunt dissection in the peri-urethral tissue plane TP. In such embodiments, the blunt edges of a dissection tool or of the implant produce blunt dissection in the peri-urethral tissue plane TP, separating the urethra U from portions of the prostate P. A penetrating tip may be used to exit the urethra U and set the depth of the penetration such that the appropriate tissue plane in the extra-urethral region can be accessed. FIGS. 1A and 1B depict the blunt tip 110 of an extra-urethral implant 100 and the tip 140 of a depth guide 150 deployed to an adjustable depth alongside the blunt tip 110. The depth guide 150 ensures delivery of the extra-urethral implant 100 beyond the urethral wall UW by creating space between the implant and the urethral wall UW. The depth guide 150 can be withdrawn at any point during the implantation of the extra-urethral implant 100. Preferably, the depth guide 150 is kept in place until the extra-urethral implant 100 has been deployed in the extra-urethral region such that the extra-urethral implant 100 mechanically displaces prostatic tissue away from the urethral lumen. The depth guide 150 can be removably fastened to the extra-urethral implant 100 by various methods. For example, the depth guide 150 can be fastened with one way tabs such that the depth guide 150 remains fixed to the extra-urethral implant 100 when the two members are pushed but can be released from in gauge meant with the extra-urethral implant 100 when the depth guide 150 is pulled proximally and the extra-urethral implant 100 is held in place. Other equivalent methods are within the scope of this disclosure.

In certain embodiments, the extra-urethral implant is delivered such that it is “wound up” like a spring prior to delivery. Upon removal of the depth guide 150, the implant 100 is configured to unwind and expand its diameter.

In certain embodiments, it is preferable to know the relative locations of the urethral sphincter and bladder with respect to prostate prior to and/or during the implantation of an extra-urethral implant. Further, it may be preferable to know where the extra-urethral implant is being deployed relative to the structures. In such embodiments, the delivery device 200 can include a transurethral imaging device. Typically, imaging devices onboard a delivery device 200 can have transmitters 210 and receivers 210 near the delivery port 220, as depicted in FIG. 2. Other embodiments can provide similar location information using optics systems. For example, in certain embodiments in which the extra-urethral implant is polymeric, the polymeric material can be chosen such that it is capable of transmitting light in addition to having desirable mechanical properties. FIG. 3 depicts the distal end 190 of the implant functions as a beacon, and can be detected by optical sensors 295 on board the delivery device. In one example, the multiple sensors 295 can track the position of the distal end 190 of the implant even though the implant is within the extra-urethral region by detecting the light emitted from the distal end 190 of the implant. By moving the distal end 290 of the delivery device, which allows multiple sensors 295 to collect light, the precise position of the implant can be determined. This kind of precise tracking and positioning can help avoid damaging sensitive parts of the local anatomy.

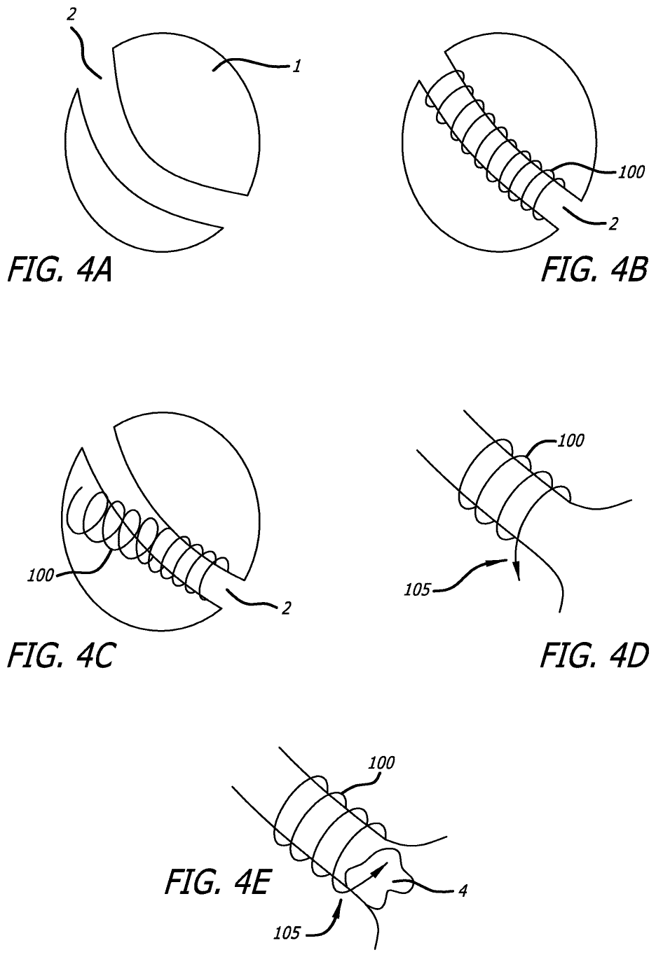

FIGS. 4A through 4E illustrate the challenges of providing a mechanically-resilient implant in the prostatic urethra. FIG. 4A depicts a view of a bend in the urethra 2. Because the implant is driven into and/or through tissue, the implant should have a sufficient degree of stiffness and strength. FIG. 4B depicts implant 100 within the extra-urethral region after having been driven through tissue. FIG. 4B depicts the urethra 2 as now straightened as compared to its previously bent condition depicted in FIG. 4A. However, over time the mechanical resilience, strength, and stiffness of the implant can have unwanted effects, as depicted in FIG. 4C. The implant in FIG. 4C has caused distortions in the prostatic tissue that in turn later caused migration of the implant 100 through tissue. Further, FIGS. 4D and 4E depict the sharp tip 105 of an implant 100 that migrates or cuts through tissue leading to erosion of tissue 4 and exposure of the implant to urine. As discussed above, chronic exposure to urine can lead to encrustation and further complications.

To accommodate this balance between the strength and stiffness needed to penetrate tissue and the flexibility and conformability needed to avoid damaging tissue in a chronic implant environment, it is preferable in certain embodiments to use a two-stage implantation process. In such a two-stage implantation, a stiff, sharp tool is advanced into tissue. Next a softer and/or less stiff implant is left behind when the delivery tool is retracted. In embodiments in which the first stage of implantation is accomplished by delivering energy to tissue, the implant can remain outside the area where energy is being delivered until implantation. In this way, the implant does not experience the delivery energy, which can be advantageous if the delivery energy would have an adverse effect on the implant.

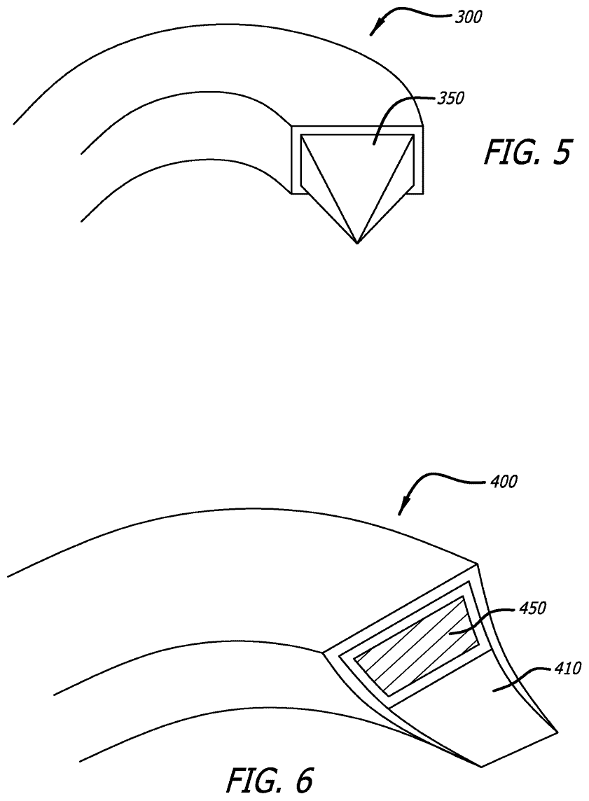

FIG. 5 illustrates a stiff delivery tool 350 and a comparatively less stiff implant 300 to be left behind when the delivery tool 350 is retracted. The core is stiff and sharp, which is preferable for driving into tissue to enable delivery of the more supple sheath implant 300 around the sharp core. Upon retraction, the comparatively less stiff sheath implant 300 is left behind as the extra-urethral implant. Preferably, the sheath has column strength substantial enough such that it does not peel back from the core when the core is being driven through tissue.

FIG. 6 illustrates another embodiment of a stiff delivery tool 400 and a comparatively less stiff implant 450 to be left behind when to delivery tool 400 is retracted. In this embodiment, the delivery tool 400 is external to the implant 450. The external delivery tool 400 is depicted as having a rectangular cross-section, but other cross-sections that facilitate directed delivery of the extra-urethral implant can also be used. The tip 410 of the delivery tool should be non-coring such that material does not build up at the tip of the delivery tool 400 and retard the progress of the delivery tool 400 and implant 450 through tissue. Advantageously, in this embodiment the implant 450 can have less column strength than the embodiment of FIG. 5 in which the implant is external to the delivery tool because the implant 450 in the embodiment of FIG. 6 is comparatively protected within the delivery tool 400. Further, the implant surface does not pass through tissue during delivery and so the implant 450 sees none of the frictional forces that the implant of the embodiment of FIG. 5 sees since it is external to the delivery tool.

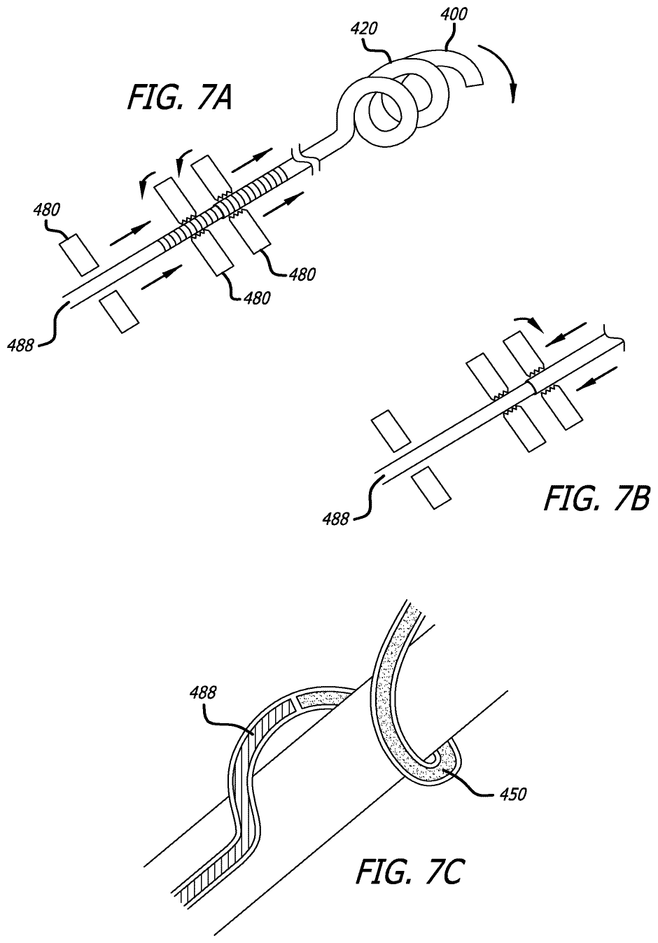

FIGS. 7A through 7C depict the delivery process of embodiments in which both the implant core 450 and the delivery tool 400 are advanced through tissue in the deployment phase. FIG. 7A depicts a helical shape for the distal end 420 of delivery tool 400. Implant core 450 is within this distal end 420 of delivery tool 400. FIG. 7A depicts lock mechanisms 480 at a region proximal to the implant core 450. These lock mechanisms 480 allow the delivery tool 400 and the implant core 450 to be manipulated together or separately by selectively locking or unlocking the delivery tool 400 and the implant core 450 with respect to each other. FIG. 7B depicts delivery school 400 being retracted while the implant pusher 488 is held fixed by some of the locking mechanisms 480. In this way, the internal implant core 450 is extruded into tissue. FIG. 7C further depicts a sectional view to illustrate the implant pusher 488 enabling delivery of the implant 450. The pusher 488 must be long enough to exit the urethra such that the implant 450 is fully embedded away from the urethral lumen, that is, the implant 450 is delivered to an extra-urethral position.

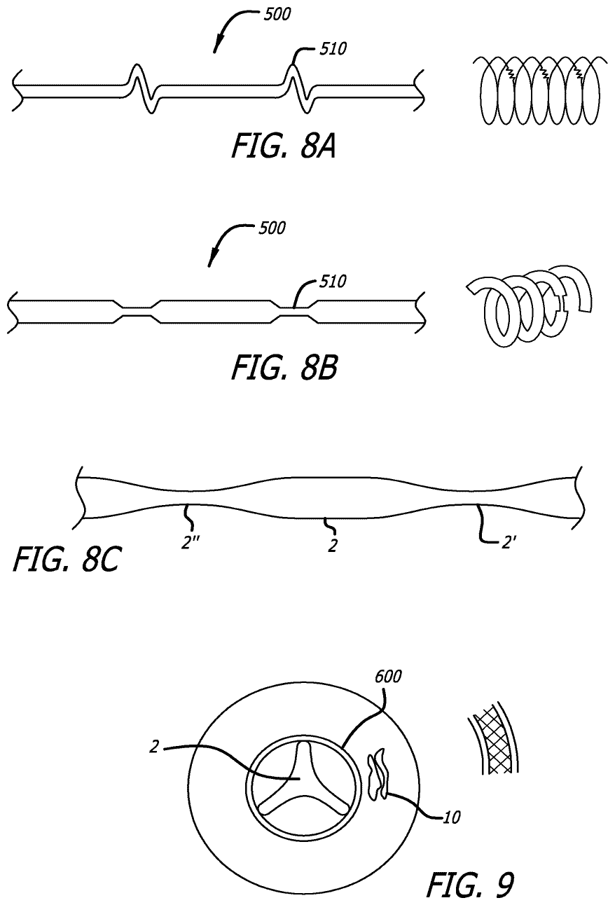

FIGS. 8A through 8C depict embodiments that can accommodate the variations of urethral anatomy while still providing the desired mechanical properties to enlarge the urethral lumen. Extra-urethral implants may need to flex longitudinally to avoid straightening the natural geometry of the urethra. Such straightening could cause discomfort and may lead to migration of the implant within tissue. FIGS. 8A and 8B depict two configurations of an embodiment of a variable strength extra-urethral implant and FIG. 8C depicts the prostatic urethra after implantation of such variable strength extra-urethral implants. As depicted in FIGS. 8A and 8B, an extra-urethral implant can have variable strength segments. Some flexible segments 510 can have hinge-like geometries to relieve longitudinal stress. The extra-urethral implant 500 can act similar to a series of independent rings rather than a straight coil. FIG. 8C depicts wider segments 2′ of the urethra 2 in which the stiffer segments of the extra-urethral implant 500 have enlarged the urethral lumen and narrower segments 2″ in which the flexible segments 510 have exerted less mechanical before action on the urethra 2. Although the entire length of the prostatic urethra has not been enlarged, it is believed that small narrowed segments coupled with a majority of large segments can still reduce or relieve symptoms of BPH.

FIG. 9 depicts a benefit of extra-urethral implants 600 that substantially encircle the urethral lumen 2. The benefit of encircling the urethral lumen is that hyperplasia subsequent to implantation of the extra-urethral implant 600 can be physically prevented from impinging on the urethral lumen. The extra-urethral implant 600 provides a physical barrier from further cell growth that narrows the urethral lumen.

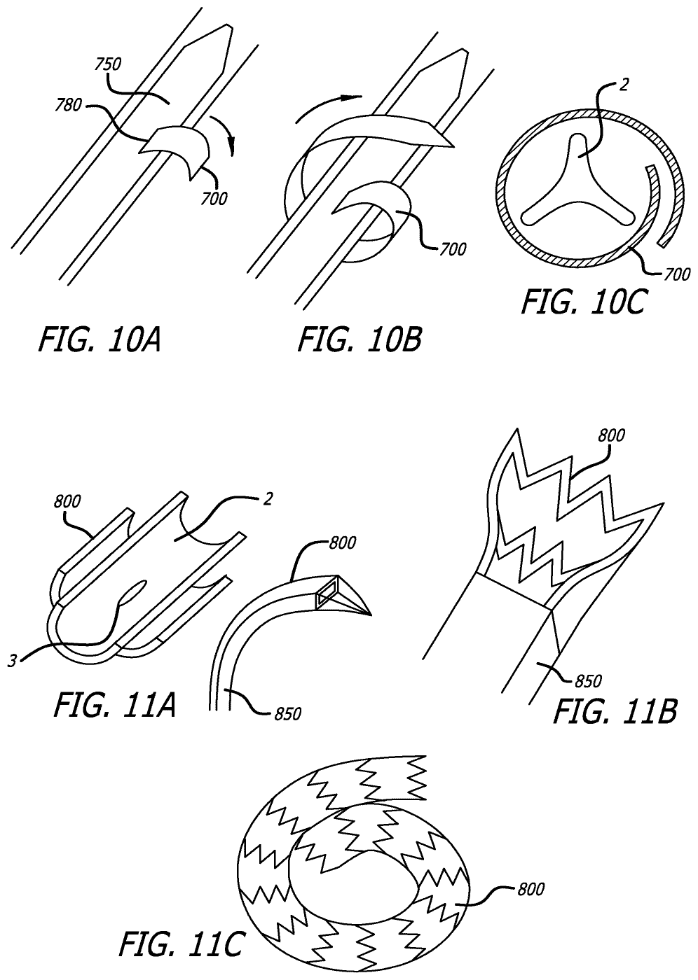

In some embodiments, controlling the extra-urethral implant as it advances longitudinally can be challenging in that it is preferable to keep the implant roughly coaxial with the lumen of the urethra. However, the urethra does not always have a straight geometry. In certain embodiments, the implant can be radially expanding, but without translating significantly along the longitudinal axis of the urethra. FIGS. 10A through 10C depicts an embodiment in which the extra-urethral implant is capable of radial expansion sufficient to displace urethral tissue, but without significant translation along the longitudinal axis of the urethra. FIG. 10A depicts delivery tool 750 within the urethral lumen and extra-urethral implant 700 advancing out of delivery port 780. Extra-urethral implant 700 has a sharp tip which enables it to advance through tissue. Extra-urethral implant 700 is sized and configured such that it provides outwardly radial force to mechanically enlarge the urethral lumen without generating significant longitudinal forces. That is, extra-urethral implant 700 is sized and configured to sufficiently circumscribe, or at least partially circumscribe, the urethral lumen. Multiple extra-urethral implants 700 can be delivered using delivery tool 752 the same prostatic urethra. Delivering multiple extra-urethral implant 700 provides enlargement of the urethral lumen along a length of the prostatic urethra. Such multiple extra-urethral implants 700 also have the benefit of avoiding the straightening problems described above.

FIGS. 11A through 11C illustrate yet another embodiment of an extra-urethral implant 800. In this embodiment, the deployment of the extra-urethral implant 800 can be in two stages. First, deployment tool 850 cuts a deployment path for the implant. Then, the implant 800 is deployed and, within the peri-urethral region, expands to a larger diameter than the deployment tool 850. FIG. 11A depicts a cross-section of the prostatic urethra 2 with entry hole 3. Extra-urethral implant 800 resides in the peri-urethral space. FIG. 11A also depicts a view of the constrained configuration of implant 800 within deployment tool 850. In FIG. 11B, extra-urethral implant 800 is shown advancing relative to the end of the deployment tool 850 and simultaneously expanding to an expanded configuration. As disclosed in other embodiments herein, extra-urethral implant 800 can be advanced relative to deployment tool 850 by various mechanisms, including but not limited to, a pusher. FIG. 11C depicts extra-urethral implant 800 in an expanded and delivered configuration. In this configuration, extra-urethral implant 800 is shown as having overlapping ends but the implant need only circumscribe enough of the prostatic urethra to create the desired mechanical enlargement of the urethral lumen.

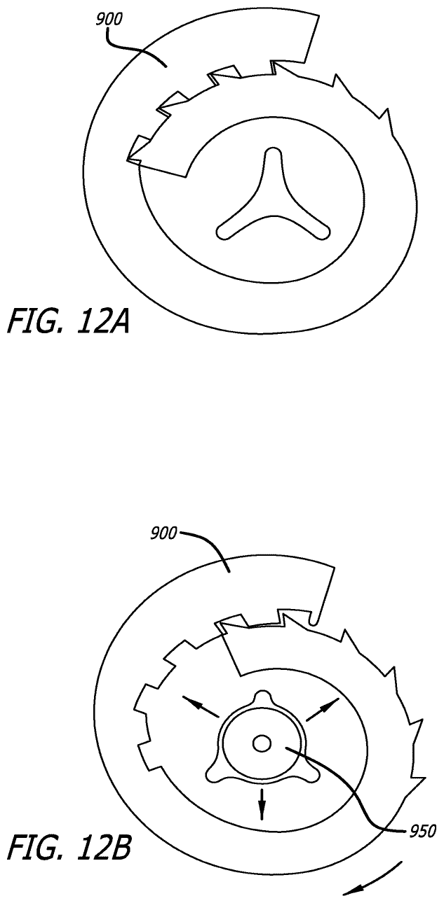

FIGS. 12A and 12B depict another embodiment of an extra-urethral implant 900. In this embodiment, the implant is frictionally connected with itself such that after initial deployment, or at a later stage, the implant could be expanded through dilation of urethra and ratcheting of the frictionally connected surfaces. FIGS. 12A and 12B depict a ring having an at least partially external loop that is in interlocking contact with an at least partially internal loop. A dilation member 950, such as a balloon, within the urethra is used to force peri-urethral tissue outward. These outward forces cause the implant to ratchet to a larger diameter. The implant will then hold peri-urethral tissue further way radially from the lumen of the urethra. This implant can be implanted initially using any of the delivery and deployment methods described herein.

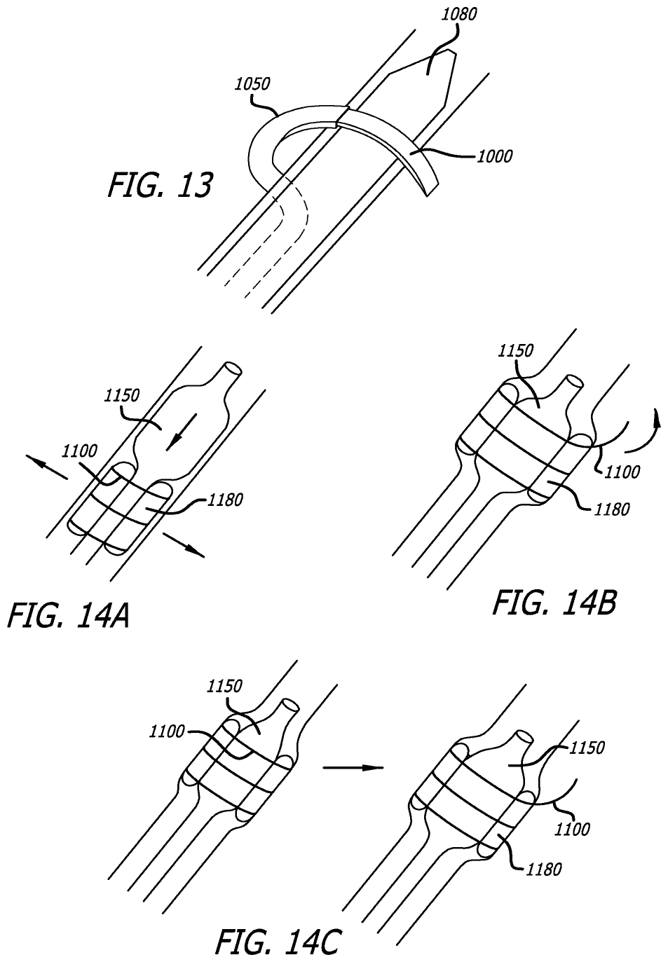

One of the challenges of expanding a narrowed urethral lumen with a coil or ring-like device is that the lumen initially has a smaller diameter and tighter radius of curvature than it will have after treatment. Some of the embodiments described herein address that challenge by expanding after deployment. FIG. 13 illustrates an embodiment in which the radius of cutting tool 1050 is tighter than the radius of extra-urethral implant 1000. Cutting tool 1050 is deployed from delivery tool 1080 and cuts into the peri-urethral space to provided deployment path for extra-urethral implant 1000. Cutting tool 1050 sets the initial deployment trajectory of the extra-urethral implant 1000, but after delivery by advancing with respect to the cutting tool 1050 the extra-urethral implant 1000 can assume greater diameter and corresponding lesser radius of curvature.

In yet another embodiment depicted in FIGS. 14A through 14C, a delivery system 1150 can be expanded at the delivery site such that the coil diameter starts deployment at the maximal urethral diameter. That is, rather than using the extra-urethral implant 1100 to expand the urethral diameter, the expansion device 1150 is used to expand the urethral diameter and the extra-urethral implant 1100 is used to maintain such expanded diameter. By expanding the deployment site, the extra-urethral implant 1100 has less diameter change post deployment. Reduced diameter change post deployment can allow for a higher degree of control over the coil trajectory. FIG. 14A shows the delivery system expanded distal to the deployment site and then moved proximally to force the distal end of extra-urethral implant 1100 into the urethral wall before the rest of the implant. By rotating the implant 1100 using members of 1180 while simultaneously pulling delivery system 1150 approximately and/or further expanding delivery system 1150, extra-urethral implant 1100 can be deployed into the peri-urethral space, as depicted in FIGS. 14B and 14C.

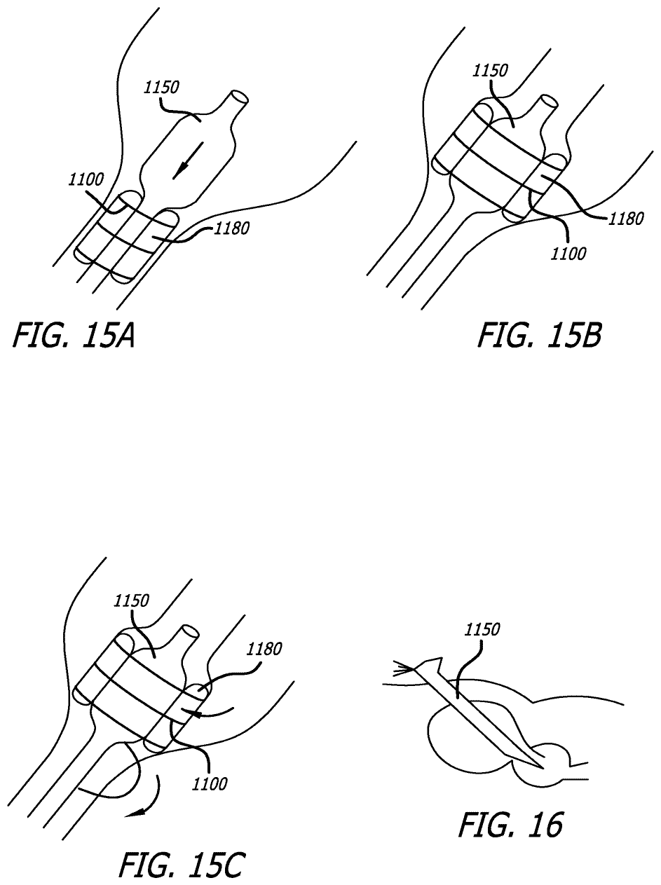

In yet another embodiment depicted in FIGS. 15A through 15C, the delivery of the extra-urethral implant 1100 could be from the bladder into the prostatic urethra. In this embodiment, the very large space of the bladder can be used advantageously to position and deliver the extra-urethral implant. The delivery process is similar to that depicted in the embodiment of FIGS. 14A through 14C. In both of these embodiments, a combination of expansion, translation, and direction of rotation works to deliver the extra-urethral implant at the desired location and diameter. Further, access could be suprapubic and into the bladder as depicted in FIG. 16. Such a delivery route can give the operator more control for delivery in the ability to avoid expanding the delivery system in situ.

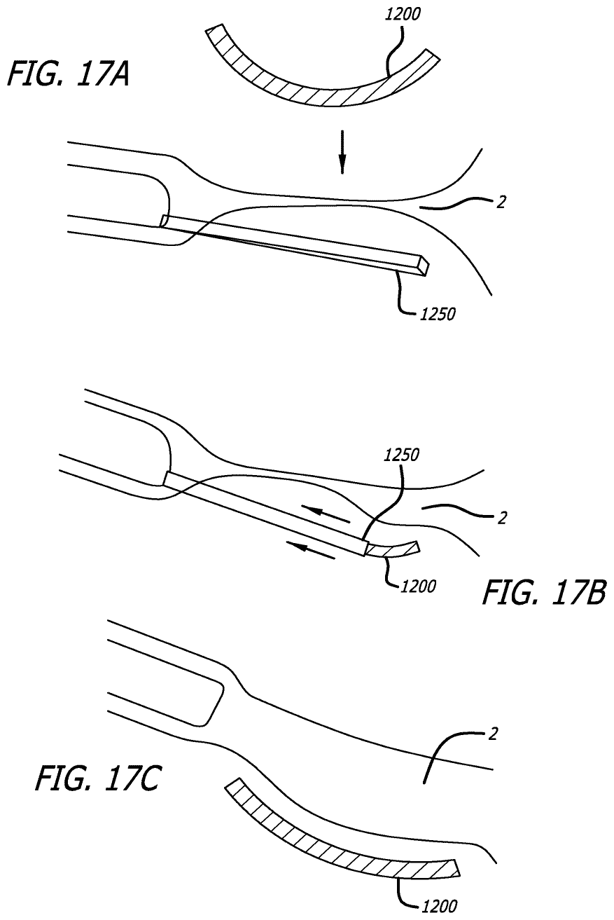

FIGS. 17A through 17C illustrate an arcuate extra-urethral implant 1200 being positioned in the peri-urethral space to enlarge the urethral lumen. The extra-urethral implant 1200 is carried within deployment tool 1250. As with other embodiments described herein, the deployment tool 1250 cuts into the peri-urethral space and the implant 1200 is advanced with respect to the deployment tool 1250. While within the deployment tool 1250, the extra urethral implant 1200 is in a substantially straight configuration. Upon deployment the extra-urethral implant 1200 can assume an arcuate configuration that helps enlarge the urethral lumen. As with other embodiments described herein, multiple implants can be positioned along the length of the prostatic urethra to achieve the desired level of enlargement.

Recalling the balance between the strength and stiffness needed to penetrate tissue and the flexibility and conformability needed to avoid damaging tissue in a chronic implant environment, other two-stage processes can be used. For example, in some subjects the tissue in the peri-urethral space can be made more mechanically resilient by a denaturing process. That is, the tissue in the peri-urethral space can be exposed to conditions that will “toughen” the tissue. Such conditions include, but are not limited to, exposure to radiofrequency heating, chemical agents, biological agents, laser energy, microwave energy, low temperatures, or equivalent means of altering the mechanical properties of tissue to cross-link portions of the tissue or otherwise stiffen the tissue. In these embodiments, the first stage can include exposure to such conditions prior to, during, or after a cutting/penetration step. Alternatively, the tissue-toughening conditions can be the first stage, and the second stage can be implantation of a self-cutting extra-urethral implant. This two-stage tissue denaturing, toughening, and/or stiffening process can be used with any of the embodiments disclosed herein or their equivalents.

Referring again to the balance between strength and stiffness needed to penetrate tissue and the flexibility and conformability needed to avoid damaging tissue in a chronic implant environment, the surface of an extra-urethral implant can be configured to enhance the mechanical coupling between the peri-urethral tissue and the implant. The peri-urethral space may contain predominantly glandular tissue that has low mechanical resilience. That is, the glandular tissue is significantly less stiff than the implant. This large mismatch in mechanical properties can be reduced by configuring the surface of the extra-urethral implant. Texture or surface features can increase the surface area contact between the implant and the prostatic tissue. Increased contact area increases the strength of the contact between the implant and the prostatic tissue. Thus, this embodiment helps overcome the mechanical mismatch between the soft, spongy prostatic tissue and the extra-urethral implant.

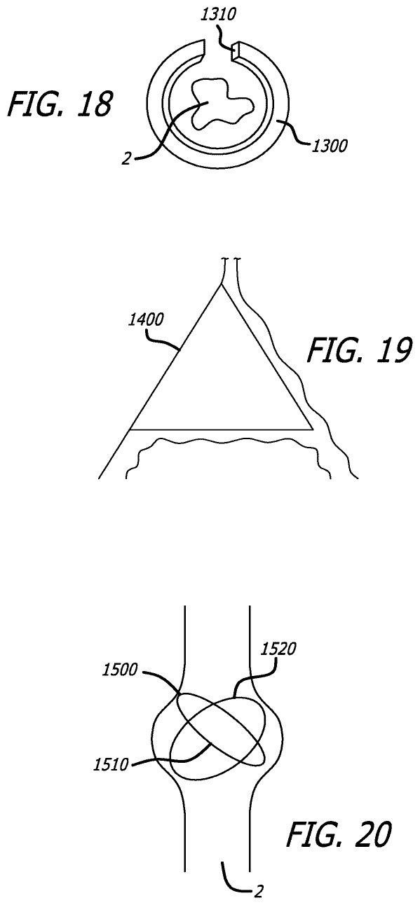

FIG. 18 depicts another embodiment of an extra-urethral implant 1300, which may be implanted by inserting the implant 1300 around a lumen, moving it circumferentially around the lumen using a sharp leading edge 1310 to penetrate tissue.

In the embodiment depicted in FIG. 19 a circumferential implant is modeled after the shape of an obstructed urethra, for instance in a triangular shape, and inserted into extra-urethral tissue. This non-circular shape can address the challenge of displacing prostatic tissue in an anatomically-tolerable way to limit migration and provide long-term implant positional stability.

In the embodiment depicted in FIG. 20, a pair of nested rings, 1510 and 1520, forms the implant 1500. The rings may be introduced in a co-planar configuration and then spread apart to widen a body lumen.

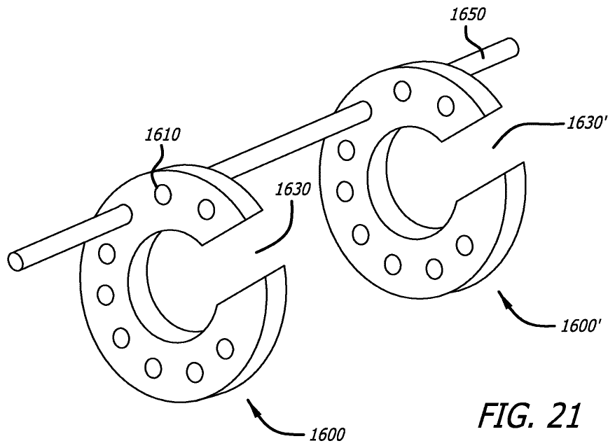

Certain embodiments of the invention include a ring that at least partially circumscribes the urethral lumen. Effective treatment of a length of prostatic urethra may require placement of multiple rings. When multiple rings are placed, one or more of the rings may shift over time and no longer provide effective opening of the urethral lumen. FIG. 21 depicts a series of rings (1600, 1600′) connected by comparatively flexible spine 1650. The spine 1650 can help prevent the rings from shifting, twisting, or otherwise moving out of position. In certain embodiments, the spine 1650 can be attached to the rings 1600, 1600′ after placement of one or more of the rings. In other embodiments, the spine is connected to the rings prior to placement of the rings. FIG. 21 depicts several holes 1610 which aid in connection the spine 1650 to the rings. FIG. 21 also depicts each ring 1600, 1600′ as having slot openings 1630, 1630′, which aid the placement of the rings about the prostatic urethra as described in embodiments disclosed herein. Slot openings 1630 and 1630′ do not have to be rotationally aligned as they are depicted in the FIG. 21 and may preferable be purposely misaligned to prevent a longitudinal segment of the prostatic urethra from being unsupported.

Certain embodiments of the invention include V-shaped extra-urethral implants. The implants are positioned such that the vertex of the V is placed outside the prostatic capsule and the legs of the V penetrate through urethral tissue and terminate in the peri-urethral space. Since the vertex is placed outside the comparatively stiffer prostatic capsule, the vertex is anchored more than it would be if it was placed within the softer tissue of the prostate gland. With the vertex placed and the prostatic capsule acting like a fulcrum, the legs of the V spread to open the angle of the V and enlarge the portion of the prostatic urethra adjacent the legs of the V. The legs can be inelastically deformed when spread by a delivery tool, such as an expanding member. Or, the legs may be compressed together during implantation and then spread when released by a delivery tool.

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art.

We claim:

1. A method for treating benign prostatic hyperplasia, comprising:
inserting an interventional device longitudinally through a urethra of a subject until at least a distal portion of the interventional device reaches a prostatic urethra, wherein the interventional device comprises a tubular elongate device with an implant;
delivering the implant to a site of the prostatic urethra, wherein the delivered implant comprises at least two ring structures configured to enlarge a lumen of the prostatic urethra, each ring structure circular in shape but forming less than a full circle, wherein the ring structures are positioned generally perpendicularly to a longitudinal axis of the urethra, and wherein each ring structure is connected to an adjacent ring structure by a segment that is generally parallel to the longitudinal axis; and
retracting the tubular elongate device after delivery of the implant, wherein the implant remains at the site of the prostatic urethra.

2. The method of claim 1, wherein the ring structures are substantially parallel to each other.

3. The method of claim 1, wherein the ring structures are configured to enlarge the lumen of the prostatic urethra by expanding radially outward relative to the longitudinal axis of the urethra.

4. The method of claim 1, wherein the implant comprises blunt ends.

5. The method of claim 1, wherein a surface of the implant does not pass through tissue as it is delivered to the site of the prostatic urethra.

6. The method of claim 1, wherein the implant is configured to flex longitudinally.

7. The method of claim 1, wherein the implant provides a physical barrier inhibiting cell growth capable of narrowing the lumen of the prostatic urethra.

8. The method of claim 1, wherein the implant is generally coaxial with the lumen of the prostatic urethra after delivery.

9. The method of claim 1, wherein the implant is configured to displace urethral tissue.

10. The method of claim 1, wherein the implant is in a substantially straight configuration during delivery.

11. The method of claim 10, wherein the implant assumes an arcuate configuration after delivery.

12. The method of claim 1, further comprising delivering at least one additional implant to the site of the prostatic urethra, wherein the additional implant also comprises at least two ring structures configured to enlarge the lumen of the prostatic urethra.

13. The method of claim 1, wherein the interventional device further comprises a transurethral imaging device.

14. The method of claim 13, further comprising viewing a position of at least a portion of the interventional device using the transurethral imaging device.

15. The method of claim 13, wherein the transurethral imaging device comprises a transmitter and a receiver.

16. The method of claim 1, wherein the interventional device further comprises an optics system.

17. The method of claim 16, wherein the implant comprises a polymeric material configured to transmit light.

18. The method of claim 17, wherein the tubular elongate device comprises one or more optical sensors configured to track a position of at least a distal end of the implant.
